(19) United States
(12) Patent Application Publication
(10) Pub. No.: US 2026/0364305 A1
(43) Pub. Date: Nov. 19, 2026

(54) DEVICE AND SYSTEM FOR THERAPEUTIC TREATMENT

(71) Applicant: CalmEnergy AS, Maura (NO)

(72) Inventor: MONICA SPORD SIGNYSDÒTTIR, Maura (NO)

(21) Appl. No.: 19/126,542

(22) PCT Filed: Nov. 2, 2023
(86) PCT No.: PCT/EP2023/080600

§ 371 Date: May 1, 2025

(30) Foreign Application Priority Data
Nov. 4, 2022 (SE) 2251281-8

(51) Int. Cl. A61M 21/02; A61H 1/02; A61M 21/00

(52) U.S. Cl. CPC A61M 21/02; A61H 1/0296; A61H 2001/0203; A61H 2201/0142; A61H 2201/1215; A61H 2201/149; A61H 2201/1607; A61H 2201/1671; A61H 2201/501; A61H 2203/0456; A61M 2021/0022

(57) ABSTRACT

A U-shaped holding device (1) comprises an outer elongate open ended U-shaped shell (2) and an inner flexible padding (3). The thickness of the padding (3) varies along the U-shape of the shell (2) and wherein the thickness of the padding (3) is essentially uniform from one end of the U-shape to the other end at each point along the U-shape. A method for applying slow and rotating movements directly to a body part of a human comprises placing the body part in an open ended U-shaped holding device (1) connected to an electric motor, rotating the holding device (1) in a first direction to a predetermined angle, rotating the holding device (1) in the opposite direction back to the starting position, rotating the holding device (1) in a second direction to a predetermined angle, and rotating the holding device (1) in the opposite direction back to the starting position.

### TECHNICAL FIELD

The present invention relates to a U-shaped holding device comprising an outer elongate U-shaped shell and an inner flexible padding and a method of providing slow movement to a body part.

### BACKGROUND

Psychologists/psychiatrists/physiotherapists and other healthcare personnel are looking for a tool that both in itself has a therapeutic effect and which also provides a foundation so that clients can more easily open up to change on another level.

With such a tool the therapist can receive several clients at the same time, and each treatment is made more efficient. There is a potential for shorter treatment times and thus faster increased quality of life and ability to work.

Also desirable, is for private individuals (children/adults) to achieve better sleep, inner calm, increased quality of life, decision-making and work ability.

This leads to increased quality of life for each individual, which in turn benefits society as a whole. Further benefits could be inner peace, stress reduction and better sleep quality in a high-speed world and reduction of physical and mental disorders. Faster recovery after mental/emotional challenges and after stroke. Restoration of balance in the digestive system. Potential tool for PTSD and paralysis.

It is a fact that we live in a society of constant stress, where changes take place faster than ever before, and with an overwhelming flow of information. Our condition today is similarly characterized by fast movements that activate the outer muscle layer, with little or no stimulation of the inner muscle layer, which is stimulated by slow movements.

At the same time, health personnel report an increasing number of burnt-out and inexplicably tired people. Stress, with the secretion of the hormone cortisol over time, is related to conditions such as concentration problems, mood swings, chest pain, failing memory, sleep problems, structural brain changes, PTSD and serious heart disease. Humans are not created for such a high load over time, where the sympathetic nervous system will inevitably be constantly activated.

Going forward, we therefore need new tools that can primarily prevent diseases, and in addition help each individual find their way back to a state of balance and optimal performance, to the benefit of society as a whole.

Systemic brain research shows that precisely slow movements with attention to sensory impressions and the breath stimulate areas in the brainstem (including the medulla oblongata), the nervous system and the interactions between the parts of the brain and between the brain and the rest of the body. In the East, it has already been known for thousands of years that slow movements, such as those found in tai chi and qi gong, are correlated with good health. Methods such as Feldenkrais and sensory motorics were later developed in the West. Later, these methods were taken apart, and the essence put together into new movement sequences. Furthermore, movement patterns such as particularly slow crawling/crabbling have also been shown to have a good effect on the aforementioned systems.

A stressed society that is moving ever faster will have an increased need for stress management methods, self-help, inner calm in each individual, shift between sympathetic and parasympathetic, especially also given the current situation in the world.

In several cases, neuromotor training has led to rapid recovery from stroke, dyslexia, stuttering, bi-polar disorders, mental exhaustion syndrome and unstable emotional life, etc.

Hence, an automatic system for which helps slow movements would be advantageous. Corresponding manual execution of the movements via another person/therapist is particularly resource- and time-consuming.

### SUMMARY

It is an object of the present invention to provide a motorized machine that performs precisely such specific, slow, rotating and pressure movements directly on people in order to increase the quality and plasticity of the nervous system, restore the balance between external and internal muscles and the balance between parts of the brain, with all the benefits this provides. Rotational movements take place around the individual body parts' own axis.

Process sequence example:
1) Focus on deep abdominal breathing to gather body and mind.
2) Grip around the head or body part.
3) Slow movement from starting position. The movement sequence always ends in the same position as it started.

Depending on the stimulus (pressure, rotation, diagonal, lift, pull), different parts of the person are affected, which in turn creates interaction between the right and left sides, the brainstem, or the limbic system.

Results: Inner peace, better sleep, stress reduction, better decision-making, increased access to unconscious information, faster retrieval after mental/emotional challenges, better function and balance in the digestive and muscular system, increased concentration, focus, faster recovery after stroke, in ME, dyslexia, stuttering etc.

Such internal, “passive” addressing and stimulation of the nervous system and internal musculature will potentially also have effects on people with paralysis or even epilepsy, which is particularly interesting to explore in a long-term perspective.

The person lies flat on a bench or on a soft floor/mat. The head is placed in a bowl-shaped module which is again attached to a base and is slowly rotated to the right and left via ball bearings or a similar solution that creates slow, steady movements.

The slow movements have the main elements of what works from qi-gong, tai-chi, Feldenkrais, and other movement meditations.

Thus, it is with good reason that movement meditations have been used in e.g. in the East far back in time. Today, systemic brain research confirms that slow movements with attention to breathing and sensory impressions stimulate areas in the brainstem. In other words, neuromotor training stimulates the nervous system and the interaction between the various parts of the brain and between the brain and the rest of the body.

Another aspect is that, unlike fast movement which stimulates the extrinsic muscles and provides mobility, slow movements mainly stimulate the intrinsic articular muscles and provide stability.

The result of the training can e.g. be increased contact with body and mind as well as providing access to unconscious information.

The movements can lead to a better balance between different aspects of oneself and create increased decision-making ability. The training can also lead to increased balance in the digestive, skeletal, and muscular systems to name a few.

According to a first aspect of the present disclosure a U-shaped holding device that comprises an outer elongate open ended U-shaped shell and an inner flexible padding is provided, wherein the thickness of the padding varies along the U-shape of the shell and wherein the thickness of the padding (3) is essentially uniform from one end of the U-shape to the other end at each point along the U-shape.

Since the padding is varied in thickness along the U-shape, the outer shell could be made with a constant radius and thus allowing for tailor made rotational speed.

According to a further aspect of the present disclosure, the shell has a cross section forming a major segment of a circle. It will be easier to control the rotational speed of the shell to a constant speed.

According to yet a further aspect of the present disclosure, the padding is thinner on the bottom of the U-shape than on the sides of U-shape. This allows for a closer fit of a human head which is, seen from above, more rectangular. Having the U-shape of the shell and a padding that is thicker on the sides and thinner on the bottom of the U-shape opposite the opening of the U-shape leaves an opening that more resembles the shape of a human head.

According to another aspect of the present disclosure, the width of the opening of the U-shape is at least 14 cm. The frontal width of a human head is generally between 14 cm and 17 cm. By having an opening corresponding to the width of a head facilitates placing the head in the holding device of the present disclosure.

A system comprising a device according to the above is according to one aspect of the present disclosure also provided. The system further comprises a support device arranged to be connected to and to rotate the holding device, the support device further comprising an electric motor arranged to act on a connection between the support device and the holding device for rotating the holding device.

The electric motor can provide basically a constant rotating speed to the holding device.

According to yet another aspect of the present disclosure, the support device comprises a gear wheel driven by the electric motor, wherein the gear wheel is arranged in contact with a toothed rack on the shell of the holding device, the toothed rack arranged on the outside of and along at least a part of the U-shape.

According to an aspect of the present disclosure, the support device comprises support wheels arranged to be in contact with the outside of the holding device.

Preferably, there are at least four support wheels arranged in a rectangle such that the holding device rests in balance on the support device.

According to yet an aspect of the present disclosure, the support device further comprises holding wheels arranged to keep the holding device in contact with the support device.

According to a further aspect of the present disclosure, the support device is connected to a control device arranged to control the speed of the electric motor. The control device has an interface that allows for setting the speed.

According to yet a further aspect of the present disclosure, the support device is connected to a control device arranged to limit the rotation angle of the holding device relative the support device. The control device has an interface that allows for setting an end rotation angle for each side.

According to an alternative aspect of the present disclosure, the support device is connected to a control device which is in wireless contact with one of in a group of interfaces comprising, an app on a smart phone, an app on a tablet, and a remote control, for allowing input for setting rotational speed and rotational angle of the holding device.

According to one aspect of the present disclosure, the system comprises the support device that is connected to the control device, wherein the control device comprises an input interface for setting various parameters comprising rotational speed and rotational angle of the holding device.

According to an aspect of the present disclosure, a method for applying slow and rotating movements directly to a body part of a human is provided. The method comprises placing the body part in a U-shaped holding device connected to an electric motor, rotating the holding device in a first direction to a predetermined angle, rotating the holding device in the opposite direction back to the starting position, rotating the holding device in a second direction to a predetermined angle, and rotating the holding device in the opposite direction back to the starting position.

Preferably, the time for rotating the holding device in the first direction to the predetermined angle is at least 3 minutes according to an aspect of the present disclosure.

According to a further aspect of the present disclosure, at least one of the predetermined angles is at least 45 degrees.

It should be emphasized that the term “comprises/comprising” when used in this specification is taken to specify the presence of stated features, integers, steps or components but does not preclude the presence or addition of one or more other features, integers, steps, components or groups thereof.

Further features of, and advantages with, the present invention will become apparent when studying the appended claims and the following description. The skilled person realizes that different features of the present invention may be combined to create embodiments other than those described in the following, without departing from the scope of the present invention.

### BRIEF DESCRIPTION OF THE DRAWINGS

The above, as well as additional objects, features and advantages of the present invention, will be better understood through the following illustrative and non-limiting detailed description of exemplary embodiments of the present invention, wherein:

FIG. 1a is a side view of a holding device according to the present disclosure.

FIG. 1b is a side view of a person with the head in the holding device.

FIGS. 2a-2c are side views of the holding device in FIG. 1 showing the holding device in various rotational angles.

FIG. 3 is a side view of a system with a holding device and a support device according to the present disclosure.

FIG. 4 is a further side view of a holding device according to the present disclosure.

FIG. 5 is a side view of a support device according to the present disclosure.

FIG. 6 is another side view of a holding device according to the present disclosure.

FIG. 7 is another side view of a support device according to the present disclosure.

FIG. 8 is a schematic view of a system of the present disclosure showing a control device.

### DETAILED DESCRIPTION OF EXAMPLE EMBODIMENTS OF THE INVENTION

The present invention will now be described more fully hereinafter with reference to the accompanying drawings, in which exemplary embodiments of the invention are shown. The invention may, however, be embodied in many different forms and should not be construed as limited to the embodiments set forth herein; rather, these embodiments are provided for thoroughness and completeness. Like reference character refer to like elements throughout the description.

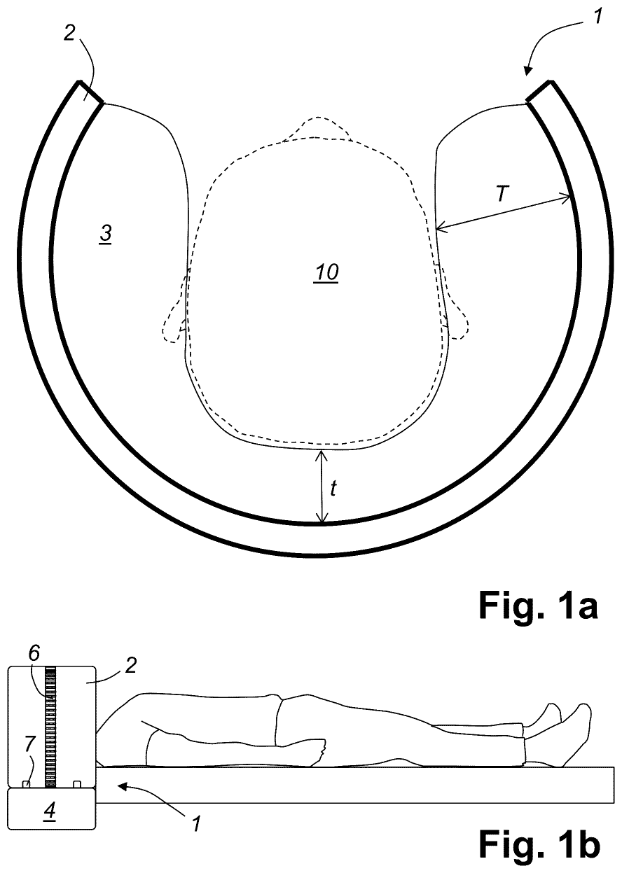

With reference to FIG. 1a, a U-shaped holding device 1 comprising an outer elongate open ended U-shaped shell 2 and an inner flexible padding 3 is shown. As can further be seen, the thickness of the padding 3 varies along the U-shape of the shell 2. As can be seen the thickness T on the sides is larger than the thickness t at the bottom of the U-shaped shell 2. In other words, the padding 3 is thinner on the bottom of the U-shape than on the sides of U-shape. However, the padding 3 thickness is essentially uniform in a direction from one open end to the other at each point in the U-shape.

An advantage of having different thicknesses along the U-shape is that there will be a softer rotation of the body part placed in the holding device 1 compared to if the rotation would follow the exact contour of the body part placed in the device. For instance, a foot placed in the holding device of the present disclosure will be rotated (up to a predetermined angle from the starting position) relatively soft compared to if the foot itself were to rotate around the heel of the foot, i.e., if a person is lying on the back with the heels contacting, e.g., a floor and the foot just falls to one side. Thus, the padding is preferably adapted such that the rotation point is rather somewhere between the heel and the toes.

FIG. 1b shows a person lying down with the head inside the U-shape. Thus, only the head is turned relative to the rest of the body. This applies of course to any body part that is placed in the holding device 1.

The same as above applies if instead a head 10 is placed in the holding device 1. Shapes of heads varies and rather than adapting the rotational speed of a specific shape, the padding is adapted such that the desired rotation point is achieved and the rotation speed when using the device can basically be constant.

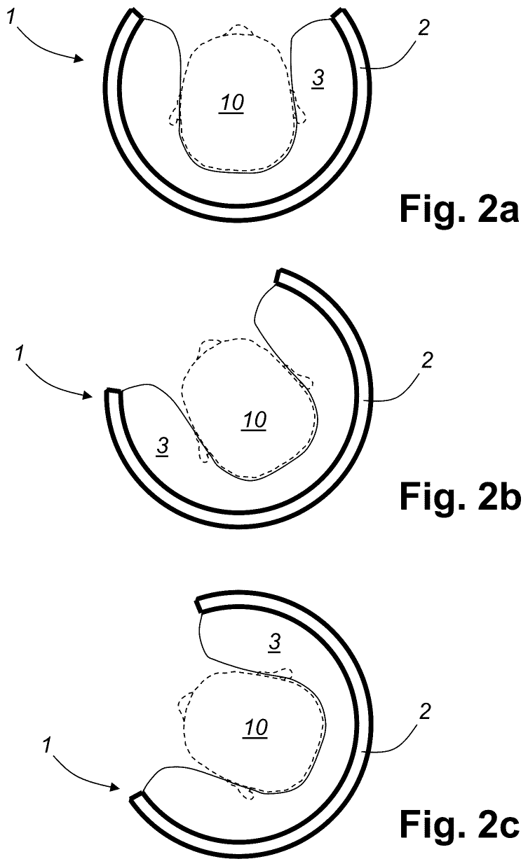

As can also be seen in FIG. 1a, the shell 2 has a cross section forming a major segment of a circle. This leads to the point of rotation being the center of the circle as is clearer from FIGS. 2a to 2c. In FIGS. 2a to 2c it is shown how the holding device 1 is rotated to the left, however, the holding device 1 can of course also be rotated to the right even though this is not shown.

For facilitating placing of a body part in the holding device 1 the opening in the U-shape should be wide enough to allow the placing in a radial direction. For instance, the width of the opening of the U-shape should be at least 14 cm, preferably larger.

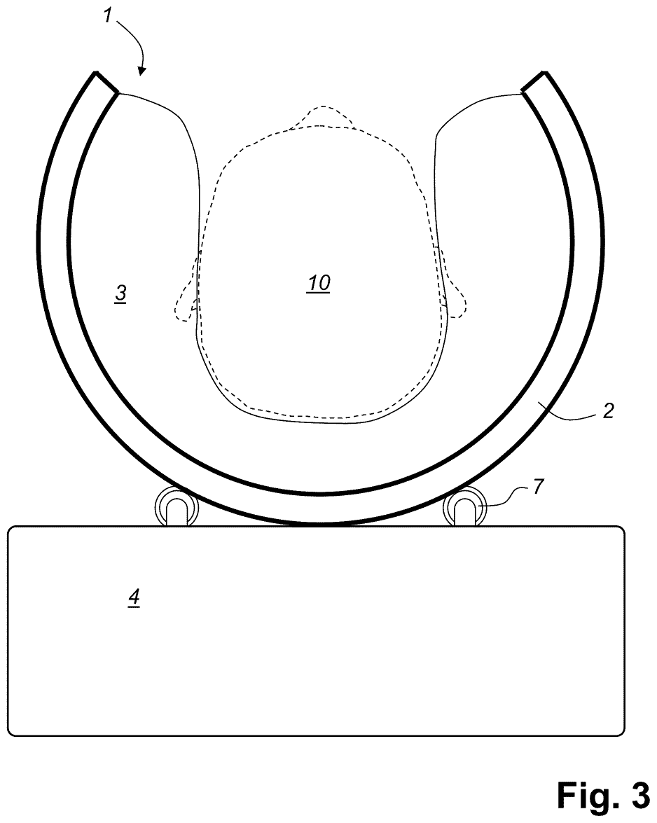

Turning to FIG. 3, a system is shown according to one embodiment of the present disclosure comprising a holding device 1 as previously described and a support device 4 arranged to be connected to and to rotate the holding device 1. The support device 4 further comprises an electric motor arranged inside the support device and arranged to act on a connection between the support device 4 and the holding device 1 for rotating the holding device 1.

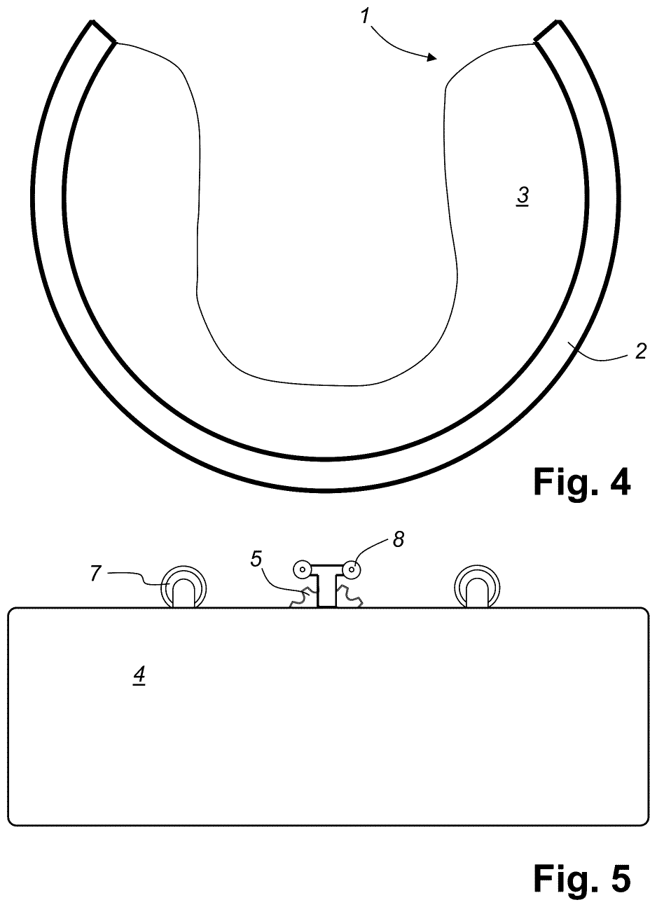

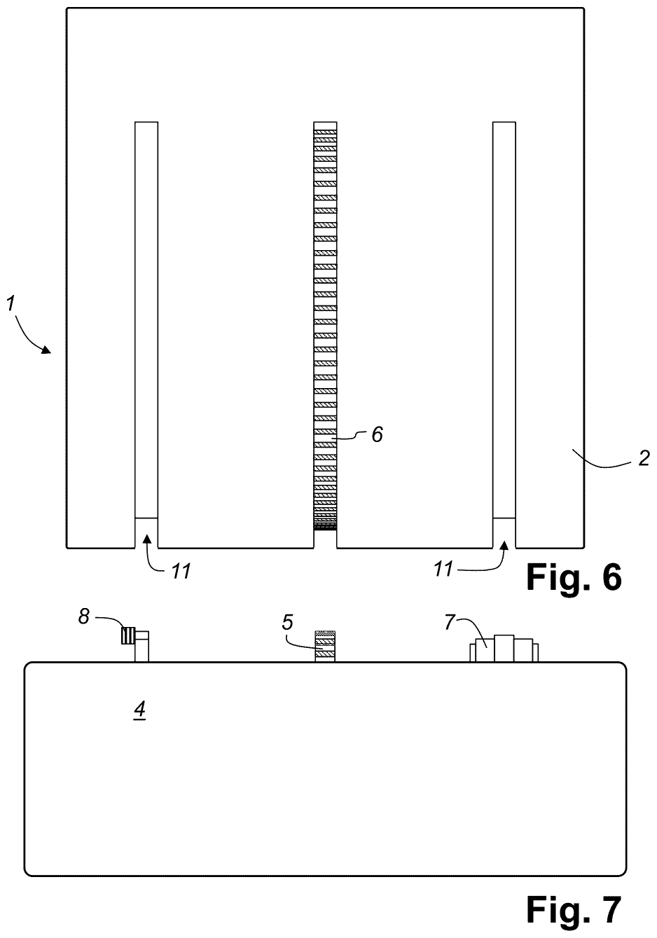

Further in FIGS. 4-7, there is shown how the support device 4 comprises a gear wheel 5 driven by said electric motor and cooperating with a toothed rack 6, see FIG. 6, to rotate the holding device 1. Thus, in assembled mode the gear wheel 5 is arranged in contact with the toothed rack 6 arranged on/in the shell 2 of the holding device 1. The toothed rack 6 shown in FIG. 6 is arranged on the outside of and along a part of the U-shape. Thus, there is a maximum point of rotation built in to the holding device 1.

FIGS. 3, 5, and 7 clearly show support wheels 7 arranged on the support device 4. FIGS. 6 and 7 show one of four support wheels 7 arranged on the support device 4 with a center part having a slightly larger diameter than the side parts on each side of the center part. The center parts of the support wheels 7 have a width corresponding to grooves 11 in the holding device 1 to control the position of the holding device 1 relative the support device 4.

Further, FIGS. 5 and 7 show the support device 4 with a holding wheel 8 arranged to keep the holding device 1 in contact with the support device 4. In the shown embodiment, there are two holding wheels 8 (only one visible) arranged at the part of the support device 4 being closest to the holding device 1, i.e., in the middle of the support device 4 and in the middle between the support wheels 7.

The shell 2 shown in FIG. 6 is composed of two bent plates, the outer plate having said grooves 11. The holding wheels 8 run on the inside of the outer plate when the holding device 1 and the support device 4 are assembled. Preferably, the holding wheels are spring biased such that they pull the holding device 1 towards the support device 4 to have a tight fit between the holding device 1 and support device 4 and minimizing the risk of derailing.

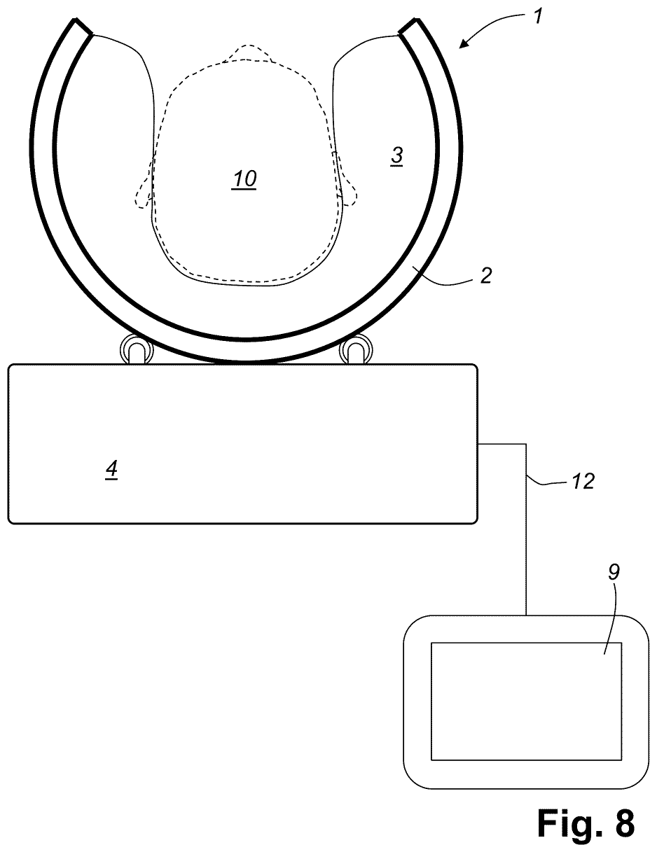

Now turning to FIG. 8, there is a connection 12 between the support device 4 and a control device 9. The control device 9 is arranged to control the speed of the electric motor and/or to limit the rotation angle of the holding device 1 relative the support device 4.

The connection 12 between the support device 4 and the control device 9 could be a wire but also wireless, wherein in the latter case there is a communications module in both the support device and the control device 9. Further, the control device 9 could also in some embodiments be in wireless contact with one of in a group of interfaces comprising, an app on a smart phone, an app on a tablet, and a remote control, in order to input data concerning rotational speed and rotational angle of the holding device 1 relative the support device 4.

In the embodiment shown in FIG. 8 of the system of the present disclosure, the support device 4 is connected to the control device 9 via a wire 12, wherein the control device 9 comprises an input interface in the form of a touch screen for setting rotational speed and rotational angle of the holding device 1.

According to the present disclosure, one way of using the system in FIG. 8 for applying slow and rotating movements directly to a body part of a human, a head 10 in FIG. 8, is first to place the head 10 in the open ended U-shaped holding device 1 connected to an electric motor inside the support device 4. Thereafter rotating the holding device 1 in a first direction to a predetermined angle, rotating the holding device 1 in the opposite direction back to the starting position, rotating the holding device 1 in a second direction to a predetermined angle, and rotating the holding device 1 in the opposite direction back to the starting position.

Typically, on cycle rotating in both directions and back to the starting position will be at least 5-6 minutes but could also be extended up to about 30 minutes. The rotation angle could be adapted to what body part should be rotated and other physical conditions but generally the predetermined angle is at least about 45 degrees.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. Furthermore, “connected” or “coupled” as used herein may include wirelessly connected or coupled. As used herein, the term “and/or” includes any and all combinations of one or more of the associated listed items.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

1. A U-shaped holding device comprising an outer elongate open ended U-shaped shell and an inner flexible padding,
wherein the thickness of the padding varies along the U-shape of the shell and wherein the thickness of the padding is essentially uniform from one end of the U-shape to the other end at each point along the U-shape.

2. The holding device, according to claim 1, wherein the shell has a cross section forming a major segment of a circle.

3. The holding device according to claim 1, wherein the padding is thinner on the bottom of the U-shape than on the sides of the U-shape.

4. The holding device according to claim 1, wherein the width of the opening of the U-shape is at least 14 cm.

5. A system comprising a holding device according to claim 1, further comprising a support device arranged to be connected to and to rotate the holding device, the support device further comprising an electric motor arranged to act on a connection between the support device and the holding device for rotating the holding device.

6. The system according to claim 5, wherein support device comprises a gear wheel driven by the electric motor, wherein the gear wheel is arranged in contact with a toothed rack on the shell of the holding device, the toothed rack arranged on the outside of and along at least a part of the U-shape.

7. The system according to claim 5, further comprising support wheels arranged on the support device.

8. The system according to claim 5, wherein the support device further comprises holding wheels arranged to keep the holding device in contact with the support device.

9. The system according to claim 5, wherein the support device is connected to a control device arranged to control the speed of the electric motor.

10. The system according to claim 5, wherein the support device is connected to a control device arranged to limit the rotation angle of the holding device relative the support device.

11. The system according to claim 5, wherein the support device is connected to a control device which is in wireless contact with one of in a group of interfaces comprising, an app on a smart phone, an app on a tablet, and a remote control, for allowing input for setting rotational speed and rotational angle of the holding device.

12. The system according to claim 5, wherein the support device is connected to a control device, wherein the control device comprises an input interface for setting rotational speed and rotational angle of the holding device.

13. A method for applying slow and rotating movements directly to a body part of a human, the method comprising:
placing the body part in an open ended U-shaped holding device connected to an electric motor,
rotating the holding device in a first direction to a predetermined angle,
rotating the holding device in the opposite direction back to the starting position,
rotating the holding device in a second direction to a predetermined angle, and
rotating the holding device in the opposite direction back to the starting position.

14. The method according to claim 13, wherein the time for rotating the holding device in the first direction to the predetermined angle is at least 3 minutes.

15. The system according to claim 13, wherein at least one of the predetermined angles is at least 45 degrees.
